(19) United States
(12) Patent Application Publication
(10) Pub. No.: US 2026/0130801 A1
(43) Pub. Date: May 14, 2026

(54) LONGITUDINAL-FEATURED COMPRESSION LEGWEAR WITH PADS TO CONTROL SYMPTOMS OF VARICOSE VEINS

(71) Applicant: Norman Noguera, Sherman Oaks, CA (US)

(72) Inventor: Norman Noguera, Sherman Oaks, CA (US)

(21) Appl. No.: 19/421,653

(22) Filed: Dec. 16, 2025

Related U.S. Application Data
(63) Continuation-in-part of application No. 19/086,009, filed on Mar. 20, 2025.
Application No. 19/086,009 is a continuation-in-part of application No. 18/941,607, filed on Nov. 8, 2024, now Pat. No. 12,521,281.

(51) Int. Cl. A61F 13/08

(52) U.S. Cl. CPC A61F 13/085

(57) ABSTRACT

Compression legwear, made to be worn by people who have varicose veins, includes at least one linear pad configured to exert a pressure of 20-30 mm Hg along a varicose vein, also includes a longitudinal opening, lateral side, for enabling a wearer to easily put on and remove the legwear.

### REFERENCE TO RELATED APPLICATIONS

This nonprovisional patent application is a CIP (“continuation-in-part”) of nonprovisional patent application bearing U.S. Ser. No. 19/086,009 (filed March 20, 2025), which is a CIP of nonprovisional patent application bearing U.S. Ser. No. 18/941,607 (filed November 8, 2024), wherein both of the above-identified patent applications are hereby incorporated by reference in their entireties for purposes of priority pursuant to Title 35 of the United States Code, Section 120.

### FIELD

The present subject matter, generally directed to compression legwear with pads to control varicose vein symptoms, is more particularly directed to compression legwear with pads for the noted purpose, which include a longitudinal feature adapted and configured to enable a user of the compression legwear to not only easily put the compression legwear on but also easily remove it.

### BACKGROUND

Compression garments—which shall be understood as articles of clothing fitting tightly around skin of a wearer, available in varying degrees of compression—include legwear providing a compression of 20-30 mm Hg or higher, which may typically require a prescription by a doctor.

A variety of compression garments are known. U.S. Pat. No. 5,179,941 to Siemssen et al. discloses contractile sleeves for peristaltic extremity treatment. U.S. Pat. No. 6,080,120 to Sandman et al. discloses a compression sleeve for gradient sequential compression systems. U.S. Pat. No. 6,254,554 to Turtzo discloses a compression sleeve to treat lymphedema. U.S. Pat. No. 6,613,007 to Reid, Jr. discloses multilayer compression stockings. U.S. Pat. No. 6,725,691 to Yakopson discloses a knitted therapeutic medical compression stocking manufactured from courses of bi-component inlaid courses of spandex yarn. U.S. Pat. No. 7,028,690 to Schneider et al. discloses a compression stocking manufactured from a knit or woven elastic fabric provided with a compression support body. U.S. Pat. No. 7,562,541 to Hermanson et al. discloses therapeutic compression stockings knitted in an integrated knit format to have an oversized heel pocket from which an ankle arch portion extends, and which is knitted in a rib stitch format that is relatively free of wrinkles when donned.

U.S. Pat. No. 10,781,542 to Gaither discloses a knit sock including a foot portion having an upper instep area and a lower sole area integrally knit of a body yarn in axial wales and circumferential courses. The lower sole area defines inner and outer arch regions. The inner arch region includes a targeted compression zone configured to reside adjacent to an inner arch of a foot of a wearer of the sock. The compression zone includes axially extending compression ridges of variable length spaced from a top portion of the compression zone to a bottom portion of the zone. When the sock is worn, a compression force applied by the foot portion within the targeted compression zone is greater than a compression force in adjacent areas of a foot portion of a sock.

NZ 710409A discloses anatomically targeted compression clothing as well as clothing items designed be worn against the skin. Items of clothing adapted to be worn against the skin comprise at least one panel adapted to provide targeted compression of at least 20% of the total length of a surface vein selected from the cephalic vein or the basilic vein in the arm; the short saphenous vein or the long saphenous vein in the leg; the collection of drainage plexuses in the buttocks and lateral aspect of the hips; or the drainage plexus in the anterior chest wall overlying the pectoral muscles; or a combination thereof. The panel mentioned, located on the inside of the clothing item, comprises a singular or plurality of protrusion(s) or pads, projecting inwardly towards the skin surface of the patient, to thereby provide a targeted compression. U.S. Pat. No. 10,172,743 to Wright et al. discloses a compression element comprising a core of fluid-filled cells as well as an outer layer of material configured for maintaining contact with skin. An embodiment comprises a folded or rolled cylindrical core of air or nitrogen-filled bubble-wrap material covered by a skin-compatible bandaging material. The compression elements described are said to provide consistent compression to a blood vessel after endovenous endothelial wall-damaging techniques. (Note: According to Wikipedia, an online information source: Endovenous laser treatment (“ELT”) is a minimally invasive ultrasound-guided technique used for treating varicose veins using laser energy commonly performed by a phlebologist, vascular surgeon, or an interventional radiologist.)

My careful assessment of the two prior art references (summarized immediately above) is as follows: They claim compression elements and/or stockings can be used to treat chronic venous insufficiency, or varicose veins. After carefully assessing these claims, I believe them to be false. For example, there are presently no known compression stockings or sleeves able to treat chronic venous sufficiency or varicose veins. Rather, compression stockings and sleeves, in my experience, are used as a conservative therapy, which means such stockings and sleeves can only provide temporary relief to wearers. Chronic venous insufficiency means varicose veins disease has existed and progressed over a length of time. As explained within this patent specification, the cause of chronic venous insufficiency in the lower extremity is valvular reflux in the saphenous veins. There is no known technology or procedure available currently to fix or treat incompetent valves in veins.

Let me be clear about the condition mentioned above: A defective or refluxing venous valve is an irreversible medical condition. Furthermore, while endovenous laser treatment is currently a procedure available to treat abnormal saphenous veins, even laser ablation cannot treat the “leaky” valves, since laser ablation treatment procedures permanently close the abnormal saphenous veins.

Indeed, the present subject matter does not claim to treat chronic venous insufficiency or varicose veins. Instead, an object of the present subject matter is to provide relief to people who suffer from chronic venous insufficiency by controlling its symptoms and providing medical-grade compression legwear designed and configured to make it tolerable and acceptable for people who may otherwise refuse to wear medical-grade legwear, despite their physician's recommendation.

Varicose veins are swollen, twisted, blood vessels, often seen bulging under the surface of the skin of a person. Varicose veins, seen as blue or purple bulges, usually appear in the legs, feet, and ankles. Varicose veins can be painful or itchy. When the veins in the legs of a person become enlarged, the valves in such veins often do not function properly and may even cause bi-directional blood flow called “valvular reflux” (in such veins). Moreover, as blood in such veins flows toward the feet, a condition called “venous hypertension” may also develop, causing unpleasant symptoms including pain, swelling, skin discoloration, and in certain worst-case scenarios, “venous ulcers.”

Graduated compression hosiery has long been used in the medical industry, for example, to aid in prevention of deep vein thrombosis and help manage post-thrombotic syndrome. Indeed, wearing graduated compression hosiery everyday may help manage varicose veins by preventing tired, swollen, generally uncomfortable legs, especially when the wearer is traveling and pregnant.

One way of controlling varicose veins of patients thus afflicted is to manage symptoms of venous valvular reflux in their legs by recommending or prescribing such patients wear special medical grade (20-30 mm Hg) firm compression stockings. However, some patients say they find such stockings difficult to put on or uncomfortable to wear, due in part to circumferential pressure inwardly directed around their legs. With traditional compression stockings, an entire length of leg must be tightly compressed to decompress the varicose veins, which may explain why people do not want to wear them. In legs, the most common veins prone to developing valvular reflux are the great and small saphenous veins, both referred to as superficial veins because they are rather close to the skin surface. Unfortunately, due to their location, the valves associated with superficial veins often fail because these valves are not protected by deep muscles and their associated tissue as are deep veins. Moreover, yet another risk factor for developing varicose veins is family history.

After endovenous radiofrequency or laser-ablation treatment, treated patients are advised to wear compression stockings primarily to prevent the risk of thrombus extending from treated saphenous veins into a deep vein. Thrombus extension, a known complication of endovenous ablation procedures, is commonly referred to as endothermal heat-induced thrombosis (“EHIT”).

The present subject matter, which is configured to deliver medical-grade pressure along treated patient saphenous veins, prevents the risk of EHIT and makes it easier for patients to wear compression stockings during recovery. Once the endothelial wall of a patient's vein is damaged by thermal heat, the treated saphenous vein will eventually close within 24 to 72 hours through blood clot formation. This is not an inherent risk, but a typical vein closure. Ultimately, the body will absorb the obliterated vein with blood clot in a matter of months.

My linear pads—when applied along treated saphenous veins—are sized and configured to ensure that diameters of varicose or dilated veins are reduced. By doing so, my linear pads will minimize the formation of blood clots in the treated veins of the patient. Another important result of using my pads is to ensure blood clots remain stable in the great saphenous vein near the saphenofemoral junction. This will prevent occurrence of EHIT in the common femoral vein after an endovenous ablation. Newly formed blood clots, referred to as acute blood clots, are soft in texture. However, my linear pads are designed and configured to be located in tiny regions adjacent to such clots and not move therefrom. Indeed, the present subject matter, generally directed to compression legwear with pads to control varicose vein symptoms, is more particularly directed to compression legwear with pads for the noted purpose, which include a longitudinal feature designed and configured for enabling a user of the compression legwear to not only easily put the compression legwear on but also easily remove it. Thus, the present subject matter not only aids in recovery and reduces inflammation; it also prevents risk of EHIT as well.

### SUMMARY

The present subject matter for compression legwear is configured to be comfortable to wear and easier for a user to put on and remove each day in comparison to known compression legwear.

Compression legwear of the present subject matter applies surface pressure to select leg regions to decompress abnormal saphenous veins located there. Also, medical-grade compression stockings of the present subject matter are manufactured from fabric and elastic materials having a strength effective to provide a surface pressure of about 15-20 mm Hg circumferentially inwardly around a leg of a person having abnormal saphenous veins. My compression stockings, made to include linear pads of the same or similar materials, apply a constant pressure to saphenous veins.

The medical-grade compression legwear of the present subject matter includes a thigh-high medical-grade compression stocking embodiment which includes three linear pads. Each pad has a width of about 5.0 centimeters (“cm”) and a thickness of about 8.0 millimeters (“mm”). A first pad extends from the upper thigh proximally to just above the knee of a wearer, medially. A second pad of the same width and thickness extends from about knee-level to adjacent an ankle, anteriorly. A third pad, of same width and thickness, extends medially from knee-level to adjacent the ankle.

The longitudinal feature of the medical-grade compression legwear of the present subject matter would also be suitable for inclusion in a knee-high medical-grade sock as well as knee-high and thigh-high medical-grade compression sleeves disclosed in U.S. Ser. No. 19/086,009 and Ser. No. 18/941,607.

For 15-20 mm Hg compression legwear, the present subject matter uses integrated linear pads to exert a surface pressure of about 20-30 mm Hg to skin where saphenous veins appear. The pads, made of a similar or an identical fabric and elastic material as the compression stocking, are designed, sized, and configured to be located atop or closely adjacent to where saphenous veins appear beneath the skin. For purposes of this patent specification, the word “pad,” when used in connection with phrase “linear pad,” shall be understood to mean a relatively thick configuration of soft material that, by exerting pressure upon a surface portion of skin, presses down upon the surface portion of skin, thereby exerting a pressure of about 20-30 mm Hg upon the skin surface portion contacted. The phrase “closely adjacent to” shall be understood to mean being spaced about 0.1-10.0 millimeters (“mm”) from the vein. When a pad is applied, e.g., to a leg, the pad changes skin surface contours near a vein. An applied pad thus increases pressure on the skin and decreases volume within surrounding soft tissue. As the pads increase pressure from about 15-20 mm Hg to 20-30 mm Hg to skin surfaces where saphenous veins appear, varicose vein diameters reduce to normal sizes. After vein-diameter reduction is achieved, the refluxing valves in saphenous veins can work normally again. This embodiment of the present subject matter—further including a longitudinal feature designed and configured to enable a user of the compression legwear to not only easily put the compression legwear on but also easily remove it—is directed to medical-grade compression stockings dimensioned and configured to be worn on the legs of a person having abnormal saphenous veins. Compression legwear, of the present subject matter, has been found to be much easier for a patient to put on and take off than conventional compression legwear and is more comfortable to wear. My medical-grade compression legwear exerts predetermined pressures to selected leg surfaces for decompressing abnormal saphenous veins present. My medical-grade compression legwear, made from fabric and elastic materials, has a strength effective to exert a surface pressure of about 15-20 mm Hg, circumferentially and inwardly, around the leg of a person having abnormal saphenous veins. My medical-grade compression legwear, including linear pads made of the same or similar materials, could add an additional surface pressure of about 15-25 mm Hg, or about 20-30 mm Hg, or about 25-35 mm Hg to skin of a patient, under which the saphenous veins are located. For informed-consent purposes, while my compression legwear embodiments may not be suitable for people with lymphedema and other non-vascular-related causes of swelling in their feet, my compression sleeve embodiments are a much better therapy solution for people who are suffering from chronic venous insufficiency for several reasons. Sleeves are easier to put on, more comfortable to wear, are less costly to manufacture, and linear pads are easier to attach.

An illustrated stocking embodiment of the present subject matter shall now be summarized.

A compression stocking has a lateral side and a medial side spaced opposite from the lateral side. The stocking comprises a compressive body configured to be worn by a person having at least one varicose vein. The stocking therefore may include at least one elongated pad secured to an inner surface portion of the medial side of the body. In embodiments, the compression stocking of the present subject matter includes at least two spaced-apart elongated pads on the medial side of the body and a pad on the posterior side. The stocking also includes a pair of spaced-apart flaps secured to spaced-apart edge margins of the compressive body along the lateral side of the body.

The stocking also includes a plurality of two-component closures spaced seriatim along the lateral side. A first component of each two-component closure is fixed to a first one of the pair of flaps and a second component of the two-component closure is fixed to a second one of the flaps.

The compressive body has an upper portion sized and designed to fit (preferably snuggly) around a thigh of a person wearing the stocking. The compressive body includes a lower portion spaced from its upper portion. The compressive body lower portion is sized, designed, adapted, and configured to receive, preferably snuggly, one of the feet of the person wearing the stocking.

One or more elongated pads, secured (either permanently or removably) to an inner surface portion of the medial side of the compressive body and at least one elongated pad, similarly secured to an inner surface portion of the posterior side of the body are configured to either overlay or be located closely adjacent to a major portion of the varicose veins of a person wearing the stocking.

Each elongated pad has opposite end portions. For one of the medial side elongated pads, one of the end portions extends to the upper thigh and the other end portion extends to above the knee. For the other medial side elongated pad, one of the end portions extends to mid-knee and the other end portion extends to about the ankle. For the posterior side elongated pad, one of the end portions similarly extends to mid-knee and the other end portion similarly extends to about the ankle. Each elongated pad is sized, designed, adapted, and configured to overlay a preselected one of a plurality of varicose veins and exert a compressive pressure of about 15-25 mm Hg, or about 20-30 mm Hg, or about 25-35 mm Hg to—or closely adjacent to—skin of a patient, under which the major portion of the one or more varicose veins of the person wearing the stocking are located.

The lateral side of the compressive body has a linear opening extending from the thigh to the foot of a person wearing the stocking. The stocking includes a pair of spaced-apart flaps secured, preferably permanently, to associated spaced-apart edge margins of the compressive body along the lateral side, so that the linear opening extends longitudinally between the spaced-apart flaps. The stocking also has a plurality of two-component closures seriatim spaced along the lateral side of the body. A first component of each closure is secured, preferably permanently, to one flap. The other component of closure is secured, preferably permanently, to the other flap. Thus, as a result, the first and second components of the plurality of closures are not only removably secured together but also seriatim spaced apart along the lateral side of the body to close the linear opening.

### BRIEF DESCRIPTION OF THE DRAWINGS

FIG. 1 is a side view of an embodiment of a medical-grade thigh-high compression stocking of the present subject matter, worn on the right leg, lateral side of, a person wearing the stocking.

FIG. 2 depicts a side view of the medical-grade thigh-high compression stocking, shown in FIG. 1, as now worn on the left leg, medial side of, the person wearing the compression stocking.

FIG. 3 is an enlarged side view of the stocking, from the region A—A, depicted in FIG. 1.

FIG. 4 presents an enlarged side view, based on FIG. 3, showing the linear opening closed.

FIG. 5 is an enlarged side view of the stocking, from the region B—B, depicted in FIG. 2.

FIG. 6 is an embodiment of a portion of elongated pad configured to be secured, temporarily or permanently, to inner surface portions, medial side of, the medical-grade compression stocking.

FIG. 7 depicts an enlarged, detail view of one of the plurality of two-component closures.

Throughout the FIGS —and the detailed description which follows—I shall use similar reference numerals, to describe and reference the similar components of the present subject matter.

### DETAILED DESCRIPTION

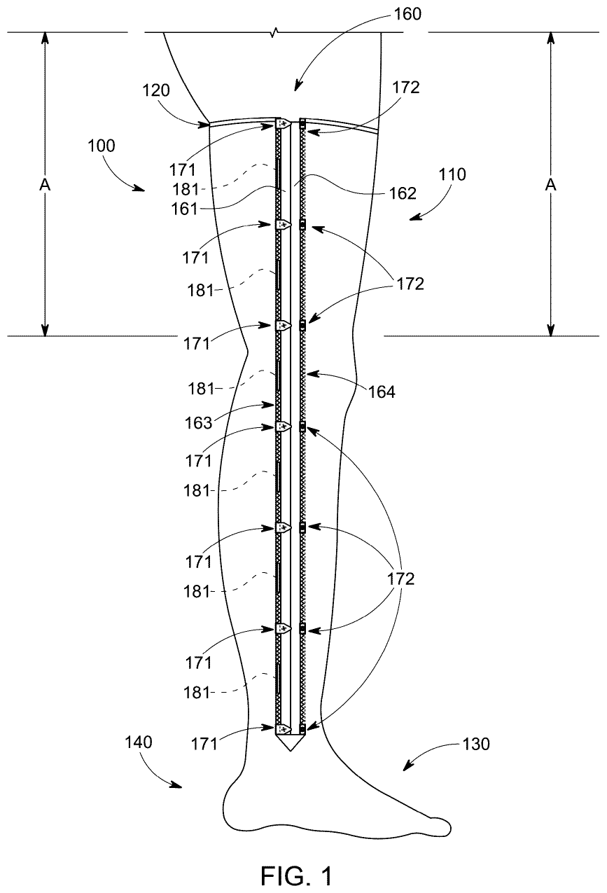

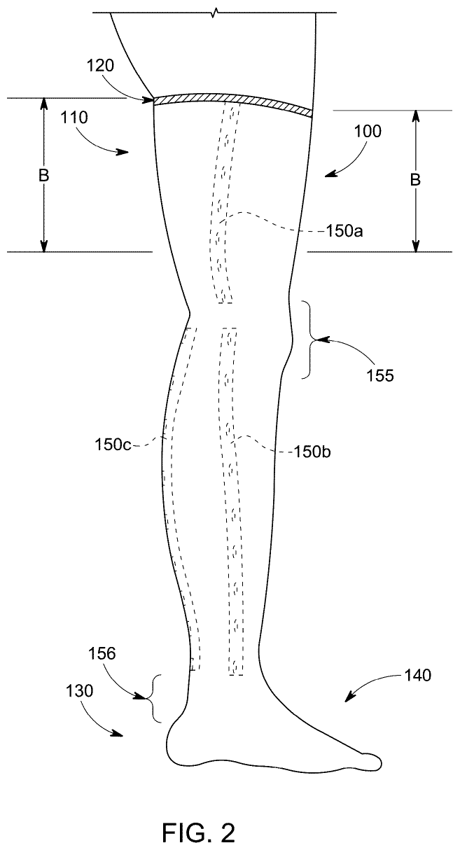

Compression stockings (FIGS. 1 and 2) of the present subject matter are sized, designed, adapted, and configured to make use of a technique that I call “targeted compression.” Applying my principles of “targeted compression,” an entire leg does not need to be tightly compressed. Instead, only those locations where the great and small saphenous veins are located are “targeted” for firm compression, by elongated pads (also called “linear pads”) secured, either temporarily or permanently, to inner surface portions of the compression stockings of the present subject matter.

The concept of targeted compression is based on a procedure involving ultrasound probe pressure, whereby varicose great and small saphenous veins decompress when a predetermined “adequate amount” of pressure is applied to preselected portions of skin over medial and posterior sides of legs—where varicose great and small saphenous veins have been located—using an ultrasound probe. The linear pads, designed into my compression footwear, serve to function like ultrasound probes, to constantly apply pressure at locations where abnormal saphenous veins are.

For purposes of the present subject matter, the term “pad” shall be understood to mean a thick piece of soft material configured to change the shape of a surface to which the pad is applied.

Therefore, whenever my pad is applied to a leg, it changes the surface contour of the skin, increasing pressure in surrounding soft tissue and decreasing its volume. When applied thusly, the pad reduces the superficial vein diameter; and when compression socks and stockings are worn, the pressure at an arc length of a leg not covered by pads is 15-20 mm Hg, while the pressure at an arc length of a leg covered by a pad of the present subject matter increases to about 20-30 mm Hg, as additional material pushes skin and soft tissue. A pressure of, for example, about 20-30 mm Hg decompresses refluxing of great and small saphenous veins, by reducing their size and enabling associated valves to work properly again. As a result, venous hypertension in lower legs is relieved.

A detailed description of the illustrated embodiment of the compression stocking 100 includes the following. The compression stocking 100 includes a lateral side (see, e.g., FIG. 1) and a medial side (see, e.g., FIG. 2) spaced about 180 degrees (about a leg of the wearer of the stocking 100) from the lateral side. The stocking 100 comprises: a compressive body 110 designed, adapted, dimensioned, and configured to be worn by a person having at least one varicose vein (not shown).

The compressive body 110 of the illustrated embodiment has an upper portion 120 sized, adapted, and configured to snuggly fit around a thigh of a person wearing the compressive body 110. Compressive body 110 also has a lower portion 130 spaced from the upper portion 120. The lower portion 130 is sized, adapted, and configured to snuggly fit a foot 140 of a person wearing the compression stocking 100. (See FIGS. 1, 2.) The compression stocking 100 of the illustrated embodiment of the present subject matter further includes at least three elongated pads 150a, 150b, and 150c (see FIG. 2) secured, either permanently or removably, to an inner surface portion of the compressive body 110. A first pad 150a of the three elongated pads of predetermined width and thickness extends medially, and approximately from the upper thigh or upper portion 120 (of the compressive body 110), to just above the knee 155 of the wearer of the compression stocking 100.

A second pad 155b (of the same width and thickness as pad 150a) of the three pads extends medially, from knee-level 155 to adjacent the ankle 156 of the wearer of the stocking 100. A third pad 150c (of the same width and thickness as pad 150a) extends anteriorly and approximately from about knee-level 155 to adjacent an ankle 156 of the wearer of the stocking 100. Each pad 150a, 150b, and 150c could be manufactured from an assortment of materials, spanning soft and firm materials and including an identical fabric and/or elastic material as the compression stocking 100. In addition, each pad 150a, 150b, and 150c could be configured to have a predetermined width, a predetermined length, and a predetermined thickness. In embodiments presented to show their utility, the pads 150a, 150b, and 150c are each configured to either overlay—or be located closely adjacent to—a major portion of a varicose vein of a person wearing the compression stocking 100.

In addition, each elongated pad 150a, 150b, and 150c is configured to overlay and exert a compressive pressure of about 15-25 mm Hg, or about 20-30 mm Hg, or about 25-35 mm Hg to—or closely adjacent to—a major portion of skin of a patient, under which varicose veins are located.

The lateral side of the compressive body 110 defines a longitudinal opening 160 (FIG. 1), which extends vertically, from the upper portion 120 of the compressive body 110 of the illustrated embodiment (adjacent to an upper portion the thigh of a person wearing the compression stocking 100), downwardly to the foot 140 of a person wearing the stocking 100. The stocking 100 also includes either a left flap 161 or a right flap 162 (see only FIG. 1) secured to spaced-apart edge margins 163 and 164 of the compressive body 110 along the lateral side of the stocking 100. Thus, the compression stocking 100 is very easy for a person to put on and remove from a leg, since the linear opening 160 extends between the spaced-apart edge margins 163 and 164 of the body 110.

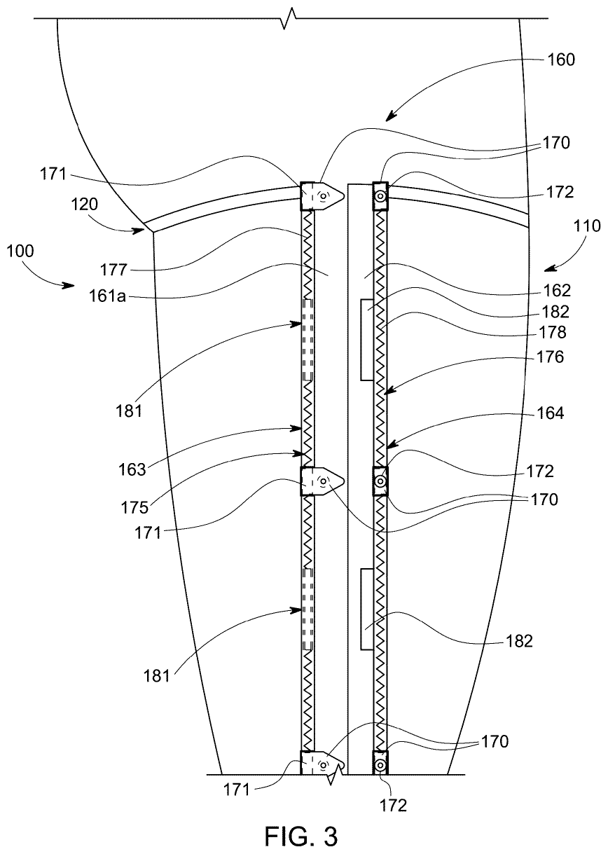

In embodiments, the compression stocking 100 of the present subject matter includes a plurality of two-component closures 170 (FIG. 3) which extend between the upper and lower ends of the longitudinal opening 160, seriatim spaced-apart, preferably equally, between the upper and lower ends of the longitudinal opening 160, along the lateral side of the compression stocking 100.

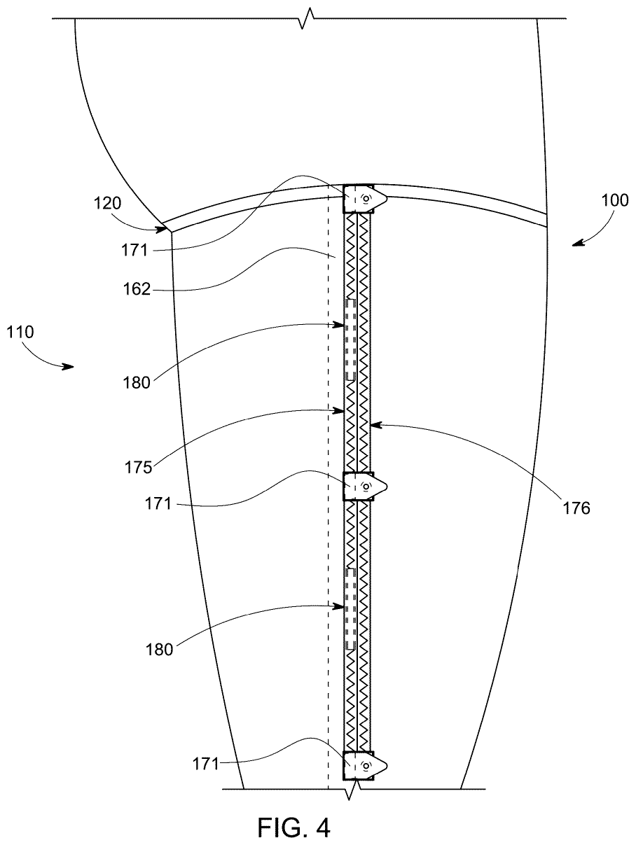

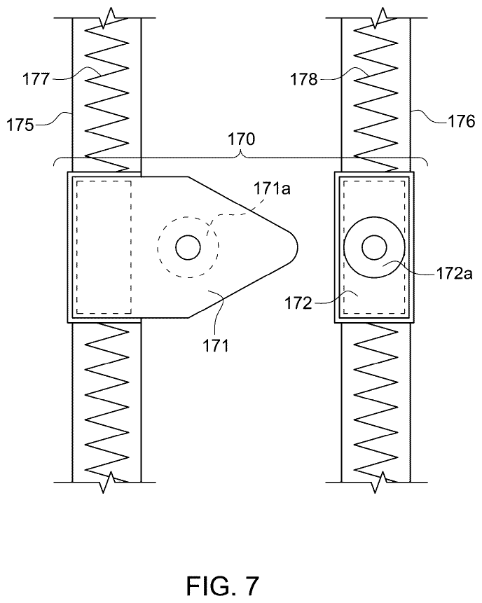

A first component 171 (FIGS. 1, 3) of each two-component closure 170 is configured to be located either adjacent to a vertically oriented edge margin of the left flap 161 (see only FIG. 1) of the right flap 162. For the embodiment shown in FIGS. 3 and 4 only the right flap 162 exists; and the region denoted by reference numeral 161a is a portion of the surface of skin of the leg of the person wearing the compression stocking 100. In this embodiment, flap 162 comprises a neoprene flap having a width of about 2 cm and a stiffness effective to extend beneath the edge margin 163. FIG. 3 presents an enlarged side view of the compression stocking 100, from the region A-A, depicted in FIG. 1. In FIG. 3, a lateral side of an upper portion of a leg of a person wearing the stocking 100 presents an upper portion of the longitudinal opening 160. Also presented in FIG. 3 are preselected lengths of elastic edging 175 and 176. In embodiments, the strips of elastic edging 175 and 176 are each attached, by zigzag stitches 177 and 178, to spaced-apart edge margins of the compressive body 110 along the length of the longitudinal opening 160. In the upper portion of the compression stocking 100 (see FIG. 3), the left elastic edging 175 and its associated zigzag stitches 177 (FIGS. 3, 7) are located on the left side of the longitudinal opening 160, while the right-side elastic edging 176 along with its associated zigzag stitches 178 (FIGS. 3, 7) are located on the right side of the longitudinal opening 160, when viewed from the lateral side of the right leg (FIG. 1) of a wearer of the compression stocking 100. To attach the edging 175 and 176 to the stocking 100, a 1-cm. (one-centimeter) wide preselected length of elastic edging 175 and 176 could be attached, preferably permanently, to the left-side of and, respectively, right-side lateral edge portions of compressive body 110 closely adjacent to the opening 160 by zigzag stitches 177 and 178. In embodiments, the first component 171 (FIGS. 1, 3) of each two-component closure 170 is fixed to seriatim spaced-apart surface portions (FIG. 3) of the preselected length of the elastic edging 175 and a second component 172 of each two-component closure 170 is similarly fixed to seriatim spaced-apart surface portions (FIG. 3) of the preselected length of the elastic edging 176.

For each two-component closure 170 (FIG. 3), to facilitate putting on and removal of the compression stocking 100 from the leg of a wearer of the stocking 100, the first component 171 defines a depression 171a (see FIG. 7), preferably circular, on its underside. The second component 172 defines an integral extension 172a, also preferably circular; and adapted and configured to be snap-engageable within depression 171a. The left-side and right-side edge margins 163 and 164 of the compressive body 110 are configured to be located proximate to the longitudinal opening 160. Preferably, the plurality of two-component closures 170 (comprising components 171, 172) could be snap-engageable tabs 171 and 172 as shown in FIG. 7, which are seriatim spaced-apart, preferably equally, along the full length of the longitudinal opening 160 of stocking 100. (FIG. 1.)

A lateral-side view of an upper leg (of a person wearing the compression stocking 100), presents the elastic edging 175, 176 spaced apart (FIG. 3) for the purpose of enabling a person to easily put on and remove a compression stocking 100; and in abutting engagement (FIG. 4) for the purpose of closing longitudinal opening. Securely attached to an underside of elastic edging 175 are a plurality of spaced-apart two-component, preferably “Velcro” (known as hook-and-loop), closure mechanisms 180 (FIG. 4). Each closure mechanism comprises a plurality of spaced-apart hook-including portions 181 (FIG. 3) and an associated plurality of loop-including portions 182. Each hook-including portion 181 is securely attached to the compressive body 110 beneath the left-side elastic edging 175. Each loop-including portion 182 is securely attached to a surface portion of the neoprene flap 162 adjacent to the right-side elastic edging 176 (FIG. 3), to form each closure 180 (FIG. 4) when the hoop-including and loop-including portions 181 and 182 are joined.

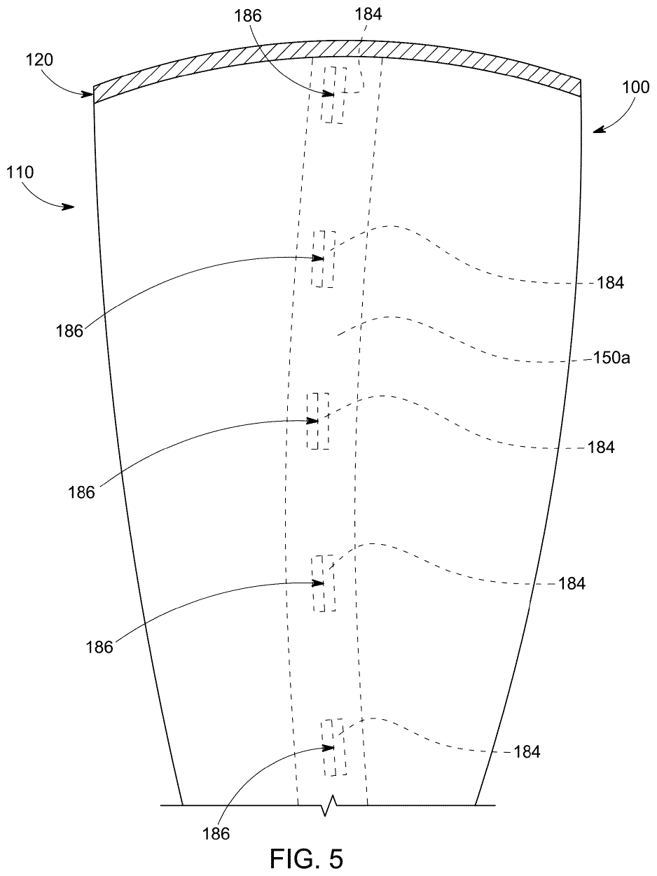

FIG. 5 is an enlarged side view of the stocking, from the region B-B, depicted in FIG. 2. In FIG. 5, an upper portion of a left leg, medial side, depicts a plurality of hooked-or-looped strips 184 configured to be spaced-apart and secured by removable stitches 186 to an inner surface of the compressive body 110. When the strips 184 are used as the hooked portion of a hook-and-loop (“Velcro”) closure mechanism, the portion of the elongated first pad 150a (in FIG. 5) would include an attached looped portion beneath each hooked portion 184 of hook-and-loop strips (in FIG. 5). The looped portions are secured, preferably removably, to first pad 150a, to enable first pad 150a to be removably secured to the interior surface of the compressive body 110 of the stocking 100.

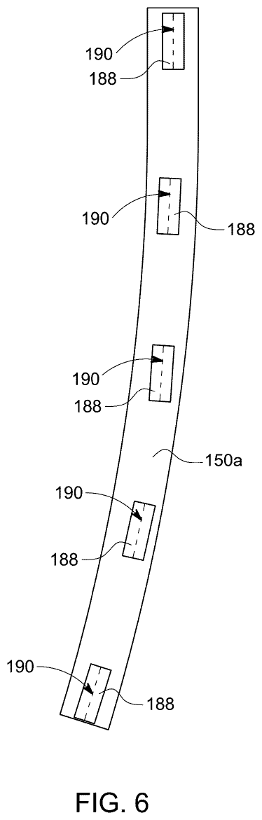

FIG. 6 depicts a portion of first elongated pad 150a configured to be secured (temporarily or permanently) to inner surface portions, medial side of, a medical-grade compression stocking 110. The portion of first pad 150a, shown in FIG. 6, would include an attached looped portion 188 beneath each hooked portion 184 of the hook-and-loop strip shown in FIG. 5. Looped portions 188 would be secured, preferably removably, to the first pad 150a by stitches 190, to enable the first pad 150a to be removably secured to inner surface portions of the compressive body 110 of stocking 100. Each of the elongated pads 150b and 150c also include looped portions 188 as well as stitches 190 to inner surface portions of the compressive body 110 of compression stocking 100.

Described in detail in this patent specification are embodiments of compression legwear including elongated pads configured for controlling symptoms of varicose veins. The legwear includes a longitudinal opening for enabling a wearer to easily put on and remove the legwear. While the present subject matter is described in detail in relation to its present embodiments, the present subject matter is not to be limited to these embodiments. On the contrary, ...many changes, alternatives, and/or modifications shall become apparent to a person of ordinary skill in the art (“POSITA”) after this patent specification and its accompanying figures have been reviewed. Therefore, alternatives, changes, and/or modifications are to be considered as constituting a part of the present subject matter insofar as they fall within the spirit and scope of the appended claims.

1. A compression stocking comprising:
a compressive body having a lateral side and a medial side spaced from the lateral side, wherein the body is configured to be worn by a person having at least one varicose vein, wherein the body defines an upper portion configured to fit around a thigh of the person and a lower portion spaced from the upper portion, wherein the lower portion is sized, adapted, and configured to fit a foot of the person wearing the stocking;
at least one elongated pad secured to an inner surface portion of the medial side, wherein the at least one pad is configured to either overlay or be located closely adjacent to a major portion of at least one varicose vein of the person wearing the stocking, wherein the at least one elongated pad has an upper end portion and a lower end portion spaced from the upper end portion, wherein the upper end portion of the pad is adapted and configured to be located proximate to an upper portion of the thigh and the lower end portion of the pad is adapted and configured to be located closely adjacent to the foot of the person wearing the stocking, wherein the at least one pad is adapted and configured to overlay and exert a preselected compressive pressure to, or closely adjacent to, said major portion of said at least one varicose vein of the person wearing the stocking,
wherein the lateral side of the compressive body includes a pair of vertically oriented and spaced-apart edge margins, wherein the compressive body edge margins define a linear opening extending from the thigh to the foot of the person wearing the stocking; and
a plurality of two-component closures spaced seriatim along the lateral side of the body, wherein a first one of the closures is fixed to a first one of the compressive body edge margins and a second one of the closures is fixed to a second one of the edge margins.

2. The stocking of claim 1, wherein the body is configured to exert a surface pressure of about 15-20 mm Hg circumferentially inwardly around the leg of the person wearing the stocking.

3. The stocking of claim 2, wherein said at least one elongated pad secured to an inner surface portion of the medial side comprises a first elongated pad adapted and configured to be secured to a first inner surface portion of the medial side, a second elongated pad adapted and configured to be secured to a second inner surface portion of the medial side, and a third elongated pad adapted and configured to be secured to a third inner surface portion of the anterior side of the stocking, wherein each of the first pad, the second pad, and the third pad is configured to exert an additional surface pressure circumferentially inwardly around the leg of the person wearing the stocking.

4. The stocking of claim 3, wherein the first elongated pad extends from between closely adjacent to the upper thigh to the knee of the wearer of the stocking, wherein the second elongated pad extends from between the knee and the ankle of the wearer of the stocking, and wherein the third elongated pad extends from between the knee and the ankle of the wearer of the stocking.

5. The stocking of claim 4, wherein the additional predetermined compressive pressure is exerted by:
the first pad upon or closely adjacent to a major portion of a first varicose vein of the person wearing the stocking;
the second pad upon or closely adjacent to a major portion of a second varicose vein of the person wearing the stocking; and
the third pad upon or closely adjacent to a major portion of a third varicose vein of the person wearing the stocking.

6. The stocking of claim 5, wherein the additional predetermined compressive pressure is about 15-25 mm Hg.

7. The stocking of claim 5, wherein the additional predetermined compressive pressure is about 20-30 mm Hg.

8. The stocking of claim 5, wherein the additional predetermined compressive pressure is about 25-35 mm Hg.

9. The stocking of claim 6, wherein the pair of vertically oriented and spaced-apart edge margins comprise a first edge margin configured to be located closely adjacent to the opening and a second edge margin is configured to be located closely adjacent to the opening and spaced from the first edge margin, wherein the stocking includes a plurality of hook-and-loop closures comprising a first associated plurality of hooked portions configured to be located closely adjacent to one of the first and second edge margins and a second associated plurality of looped portions configured to be located closely adjacent to the other one of the first and second edge margins.

10. The stocking of claim 7, wherein the pair of vertically oriented and spaced-apart edge margins comprise a first edge margin configured to be located closely adjacent to the opening and a second edge margin is configured to be located closely adjacent to the opening and spaced from the first edge margin, wherein the stocking includes a plurality of hook-and-loop closures comprising a first associated plurality of hooked portions configured to be located closely adjacent to one of the first and second edge margins and a second associated plurality of looped portions configured to be located closely adjacent to the other one of the first and second edge margins.

11. The stocking of claim 8, wherein the pair of vertically oriented and spaced-apart edge margins comprise a first edge margin configured to be located closely adjacent to the opening and a second edge margin is configured to be located closely adjacent to the opening and spaced from the first edge margin, wherein the stocking includes a plurality of hook-and-loop closures comprising a first associated plurality of hooked portions configured to be located closely adjacent to one of the first and second edge margins and a second associated plurality of looped portions configured to be located closely adjacent to the other one of the first and second edge margins.

12. The stocking of claim 5, wherein each closure of the plurality of two-component closures comprises a seriatim spaced-apart pair of tabs, wherein the tabs are snap-engageable together.

13. The stocking of claim 12, wherein the additional predetermined compressive pressure is about 15-25 mm Hg.

14. The stocking of claim 12, wherein the additional predetermined compressive pressure is about 20-30 mm Hg.

15. The stocking of claim 12, wherein the additional predetermined compressive pressure is about 25-35 mm Hg.

16. The stocking of claim 13, wherein the pair of vertically oriented and spaced-apart edge margins comprise a first edge margin configured to be located closely adjacent to the opening and a second edge margin is configured to be located closely adjacent to the opening and spaced from the first edge margin, wherein the stocking includes a plurality of hook-and-loop closures comprising a first associated plurality of hooked portions configured to be located closely adjacent to one of the first and second edge margins and a second associated plurality of looped portions configured to be located closely adjacent to the other one of the first and second edge margins.

17. The stocking of claim 14, wherein the pair of vertically oriented and spaced-apart edge margins comprise a first edge margin configured to be located closely adjacent to the opening and a second edge margin is configured to be located closely adjacent to the opening and spaced from the first edge margin, wherein the stocking includes a plurality of hook-and-loop closures comprising a first associated plurality of hooked portions configured to be located closely adjacent to one of the first and second edge margins and a second associated plurality of looped portions configured to be located closely adjacent to the other one of the first and second edge margins.

18. The stocking of claim 15, wherein the pair of vertically oriented and spaced-apart edge margins comprise a first edge margin configured to be located closely adjacent to the opening and a second edge margin is configured to be located closely adjacent to the opening and spaced from the first edge margin, wherein the stocking includes a plurality of hook-and-loop closures comprising a first associated plurality of hooked portions configured to be located closely adjacent to one of the first and second edge margins and a second associated plurality of looped portions configured to be located closely adjacent to the other one of the first and second edge margins.

19. The stocking of claim 16, wherein the plurality of two-component snap-engageable tabs are configured to be seriatim spaced apart along the length of the opening whereupon, when the plurality of tabs are snap engaged, the first and second edge margins abut for closing the opening.

20. The stocking of claim 17, wherein the plurality of two-component snap-engageable tabs are configured to be seriatim spaced apart along the length of the opening whereupon, when the plurality of tabs are snap engaged, the first and second edge margins abut for closing the opening.
